(19) United States
(12) Patent Application Publication
(10) Pub. No.: US 2014/0093598 A1
(43) Pub. Date: Apr. 3, 2014

(54) COMPOUNDS AND EXTRACTS FROM GNETUM AFRICANUM AND ANTI-INFLAMMATORY ACTIVITY RELATED THERETO

(71) Applicants: Carole Robert, Blainville (CA); Sue Wehner, Ste-Marthe (CA)

(72) Inventors: Carole Robert, Blainville (CA); Sue Wehner, Ste-Marthe (CA)

(73) Assignee: PHARMAFRI-CAN INC., Levis, QC (CA)

(21) Appl. No.: 13/812,427

(22) PCT Filed: Jul. 26, 2011
(86) PCT No.: PCT/CA2011/000871

§ 371 Date: Nov. 1, 2013

Related U.S. Application Data
(60) Provisional application No. 61/367,567, filed on Jul. 26, 2010.

(51) Int. Cl. A61K 36/17; A61Q 19/00; A61K 8/60; A61K 31/7034; A61K 8/97

(52) U.S. Cl. CPC A61K 36/17; A61K 31/7034; A61K 8/97; A61K 8/602; A61Q 19/00
USPC 424/779; 424/725; 514/35

(57) ABSTRACT

The invention relates to extracts and compositions from Gnetum africanum and methods for obtaining the extracts, wherein the extracts comprise resveratrol and have anti-inflammatory activity. Methods of using the extracts for the prevention and/or treatment of inflammatory diseases, and compositions which comprise the extracts including cosmetic compositions are also encompassed.

### FIELD OF THE INVENTION

The invention relates to the field of natural products. More particularly, it concerns compounds and extracts from Gnetum africanum having anti-inflammatory activities. It also concerns compositions and methods for the prevention and/or treatment of inflammation reactions in humans and animals.

### BACKGROUND OF THE INVENTION

Chronic inflammation is strongly related to many diseases and conditions, which can present as acute inflammation as part of allergic responses or infections or as chronic idiopathic conditions, such as arthritis. Inflammatory responses can occur as topical eruptions of the skin (for example, reactions to poison ivy) or internally in the joints (for example, in arthritis), organs and muscles.

The medicinal attributes of plants and their extracts have been well known since ancient times. Evidence that plant-derived compounds are taking on a more significant role in the pharmaceutical market today is the U.S. Food and Drug Administration's official new guidance for approvals of botanical drugs. This guidance was published in 2004 and the first botanical drug approval was granted in 2006.

Gnetum africanum is a popular vegetable commonly found in tropical Africa. It is collected in the wild (rather than cultivated) and it is added to foods and made into fusions. Although traditional African medicine includes a few medical uses for Gnetum africanum, there is no clear scientific evidence of any therapeutic efficacy for that plant. Furthermore, the conditions under which under which it would be possible to obtain reliable medicinal or cosmetic products derived from that plant have not been properly documented.

Because currently available anti-inflammatory drugs are frequently associated with severe adverse reactions, there is a need for compounds, compositions, and drugs for the prevention and treatment of inflammatory diseases, such as rheumatoid arthritis, rheumatic fever, many respiratory conditions, allergies, joint and muscle injuries, etc.

There is also a need for botanical and plant-derived compounds and extracts having desirable pharmaceutical properties, more particularly the prevention or treatment of inflammation.

There is also a need for biologically active extracts from Gnetum africanum and methods for obtaining the same.

The present invention addresses these needs and other needs as will be apparent from review of the disclosure, figures and description of the features of the invention hereinafter.

### BRIEF SUMMARY OF THE INVENTION

The present inventors have found compounds and extracts deriving from Gnetum africanum having anti-inflammatory activities.

According to one aspect, the invention relates to a plant extract possessing anti-inflammatory activity and which is derived from Gnetum africanum.

The invention further relates to purification methods for obtaining a plant extract having an anti-inflammatory activity. In one embodiment the method comprises: (i) providing plant material from Gnetum africanum; (ii) submitting the plant material to a solvent extraction; and (iii) removing the solvent.

In another embodiment the purification method comprises the steps of: (i) grinding plant material (preferably previously dried) from Gnetum africanum; (ii) extracting the ground plant material with one or more suitable solvents to obtain a solvent extract; and (iii) recovering and drying the solvent extract to obtain the plant extract.

The invention further relates to compositions comprising a plant extract as defined herein and/or a plant extract obtained by any of the purification methods herein; in combination with a suitable carrier, diluent or excipient.

The invention also concerns resveratrol glycosides. In one embodiment the resveratrol glycoside consists of a compound identifiable under detection conditions of Table 1 by the presence of a peak having a retention time of 9.1 min on a HPLC-APCI-MSD chromatogram. In another embodiment the resveratrol glycoside consists of a compound identifiable under detection conditions of Table 1 by the presence of a peak having a retention time of 9.5 min on a HPLC-APCI-MSD chromatogram.

Another related aspect of the invention concerns the use of a compound or extract as defined herein for the manufacture of pharmaceutical compositions and drugs. One particular example is an anti-inflammatory composition comprising a plant extract from Gnetum africanum. In embodiments, the extract comprises at least about 0.002% w/w, resveratrol and/or one or more resveratrol glycosides. In another embodiment the extract comprises a resveratrol glycoside as defined herein.

Accordingly, in some aspects the compound(s) or extract(s) according to the invention are used for the manufacture of medicaments, and more particularly medicaments useful in the prevention and/or treatment of inflammation.

A related aspect concerns a method for a method for the prevention and/or treatment of an inflammatory disease. The method generally comprises administering to a subject in need thereof compound(s), extract(s), and/or composition(s) as defined herein.

The invention also encompasses cosmetic compositions comprising compound(s), extract(s), and/or composition(s) as defined herein.

An advantage of the present invention is that it provides extracts and compositions deriving from Gnetum africanum which possess desirable anti-inflammatory activity. Advantageously also, plant extracts obtained according to the invention have an elevated concentrations of resveratrol and comprises novel resveratrol glycosides.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

FIG. 1 is a line graph showing a HPLC-DAD profile of sample #E-01 (leaves from the Equatorial forest) obtained at the monitoring wavelength of 325 nm. Resveratrol is the major peak.

FIG. 2 is a line graph showing an ion chromatogram of HPLC-APCI-MSD profile of an ethanolic extract of sample #E-47 (leaves from the Equatorial forest). Two overlaid lines are represented, one of 229 [M+H]⁺ and the other of 391 [M+ glyc]⁺. In addition to resveratrol (peak #3), two glycosides (peak #1 and peak #2) are visible.

FIG. 3A and 3B are line graphs showing a dose response effect of resveratrol (1 μM-100 μM) and G. africanum extracts (25-200 μg/ml) on LPS induced TNF-α production in RAW 264.7 cells. RAW cells were treated with resveratrol (FIG. 3A) or G. africanum extracts (FIG. 3B) for 1 hour and then induced with LPS overnight. Cell culture supernatants were analyzed for TNF-α production by ELISA.

FIG. 4 is a bar graph showing the dose response effect of G. africanum leaf and stem extracts (200 μg/ml) on LPS induced TNF-α production in RAW cells. RAW 264.7 cells were treated with leaf or stem extracts for 1 hour and then induced with LPS for overnight. Cell culture supernatants were analyzed for TNF-α production by ELISA.

FIG. 5 is a line graph showing a dose response effect of methanol, ethyl acetate and ethanol G. africanum stem extracts (25-200 μg/ml) on LPS induced TNF-α production in RAW cells. RAW 264.7 cells were treated with stem extracts for 1 hour and then induced with LPS for overnight. Cell culture supernatants were analyzed for TNF-α production by ELISA.

### DETAILED DESCRIPTION OF PREFERRED EMBODIMENTS

Unexpectedly, the present inventors have found that compounds and extracts deriving from Gnetum africanum have anti-inflammatory activities. Accordingly, composition comprising compound(s) or extract(s) deriving from Gnetum africanum can be useful for the prevention and/or treatment of inflammation.

### Plant Extracts

One aspect of the present invention relates to a plant extract having an anti-inflammatory activity and which is derived from Gnetum africanum.

As used herein, the “anti-inflammatory activity” refers to the property of a substance or composition in reducing acute and/or chronic inflammatory responses, and/or in preventing or treating an inflammatory-related disease. The term “inflammatory-related disease” refers to any and all abnormalities associated with chronic or acute inflammation including, but not limited to, immune mediated inflammatory diseases (IMID) and autoimmune diseases such as arthritis, erythematosus (SLE), glomerulonephritis, vasculitis, psoriatic arthritis, systemic lupus erythematoses (SLE), idiopathic thrombocytopenic purpura (ITP), psoriasis, Crohn's disease, inflammatory bowel disease, ankylosing spondylitis, Sjögren's syndrome, Still's disease (macrophage activation syndrome), uveitis, scleroderma, myositis, Reiter's syndrome, and Wegener's syndrome.

As used herein, the term “plant extract” refers to a concentrated preparation of a plant material obtained by isolating or purifying desired active constituents with one or more extraction techniques. Examples of extraction techniques include, but are not limited to, solvent extraction (e.g. ethanol, methanol, ethyl acetate), chromatography and the like. For the purpose of clarification, infusions and other similar concoctions of Gnetum prepared according to African traditional medicine are not considered a “plant extract” according to the invention, and as such, are exclude from the scope of the invention.

Preferably the plant extract derives from the plant Gnetum africanum. As used herein, the term “derives from” or “derived from” refers to a product, compound or composition which is(are) obtained from, or can be traced back, to a given definite source. A particular example according to the invention includes resveratrol extracted or isolated from Gnetum africanum.

As used herein, the terms “Gnetum africanum”, “G. africanum” or “Gnetum” refers to the plant which commonly grows in tropical Africa. The plant is commonly called “mfumbwa” (kikongo), “longongia” (lingala) and “banvale” (Azande). Many different varieties are known, including the Asusutan, the Oron, the Ikom, the Welw and the Koko variety. Various sources of Gnetum africanum can be used according to the invention, including Gnetum from the Mayombe forest the equatorial forests of Congo. In preferred embodiments, the plant extracts according to the invention are obtained from the Gnetum africanum plants growing in the equatorial forests of Congo. Preferably, the leaves, stems and/or roots are used for obtaining a plant extract according to the invention but it is conceivable that other plant tissues such as flowers, fruits, tubers, corms, etc. may also be used. The invention further encompasses the plant Gnetum bucholzianum, and processed materials, extracts and resveratrol glycoside therefrom.

Those skilled in the art know how to identify useful sources of a plant material or plant extracts and how to measure levels of anti-inflammatory activity in different types of extracts or compositions. Anti-inflammatory activity can be measurable by various in vivo, in vitro, ex vivo and/or in situ techniques, including for instance the in vitro assay using RAW cells described hereinafter in the Examples section.

Similarly, Example 4 provides detailed analyses of the content of various biochemicals (e.g. lipids, proteins, sugars, et.) in selected parts of Gnetum africanum. Those analyses could provide supporting data in assessing whether a plant material or plant extract is derived from Gnetum africanum.

In some embodiments the extracts and compositions of the inventions comprise resveratrol. As used herein, the term “resveratrol” refers to the compound having the chemical formula C₁₄H₁₂O₃. Resveratrol is also defined by the CAS Registry Number 501-36-0 and known under other names, including, but not limited to, trans-resveratrol, trans-3, 5,4′-Trihydroxystilbene; 3,4′,5-Stilbenetriol; (E)-5-(p-Hydroxystyryl)presorcinol; and (E)-5-(4-hydroxystyryl)benzene-1,3-diol.

In various embodiments the plant extract may comprise from at least about 0.002% w/w, 0.003% w/w, 0.004% w/w, 0.005% w/w, 0.075% w/w, 0.01% w/w, or more resveratrol. In various embodiments, the plant extract comprises at least about 0.0025% w/w, 0.075% w/w, 0.01% w/w, 0.02% w/w, 0.05% w/w, 0.1% w/w, 0.5% w/w, 1% w/w or more resveratrol.

The plant extract may also comprise 20 μg, 30 μg, 40 μg, 50 μg, 75 μg, 100 μg or more resveratrol per gram or ml of extract. In various embodiments, the plant extract comprises from at least about 25 μg/g, 50 μg/g, 75 μg/g, about 100 μg/g, about 250 μg/g, about 500 μg/g, about 1000 μg/g, about 1500 μg/g, about 2000 μg/g, about 2500 μg/g, about 3000 μg/g, about 4000 μg/g, about 5000 μg/g, about 10 000 μg/g, about 15 000 μg/g, or at least about 16 500 μg/g resveratrol.

The present inventors have further identified new resveratrol glycosides referred to herein as “resveratrol glycoside-1” and “resveratrol glycoside-2”. As described hereinafter at Example 1 and more particularly in Table 1, resveratrol glycoside-1 and resveratrol glycoside-2 are identifiable as distinct peaks by HPLC-MSD. More particularly, on a HPLC-APCI-MSD chromatogram and under the detection conditions specified in Table 1, resveratrol glycoside-1 is identifiable by the presence of a peak having a retention time of 9.1 min, whereas resveratrol glycoside-2 is identifiable as a peak a retention time of 9.5 min. For comparison, resveratrol has a retention time of 10.1 min. Table 1 further provides mass spectrometric fragmentation pattern for identification of resveratrol glycoside-1 and -2.

Accordingly, an additional aspect of the invention concerns resveratrol glycoside-1 and resveratrol glycoside-2 in an isolated or purified form. Preferably the resveratrol glycoside-1 and -2 are isolated or purified from a plant, more preferably from Gnetum africanum. The invention further encompasses compositions (e.g. pharmaceutical compositions) comprising at least one of glycoside-1 and resveratrol glycoside-2.

Another aspect of the invention further concerns a plant extract having a measurable anti-inflammatory activity, the extract comprising resveratrol glycoside-1, resveratrol glycoside-2, or both. The plant extract may further comprise lipids (e.g. omega-6, omega-3, saturated and trans fats), minerals (e.g. Mg, Mn, Se, P, etc.), soluble and insoluble fibers, sugars (e.g. glucose), vitamins (e.g. vitamin E) and amino acids and proteins. Table 3 herein after provides a non limitative list of constituents which can be found in Gnetum africanum.

### Methods of Extractions

Those skilled in the art will readily appreciate that plant materials, plant extracts, resveratrol glycosides and/or compositions of the invention can be readily obtained by using various processing, and/or purification methods and techniques. The plant extracts, and/or resveratrol glycosides may be prepared by extracting, blending, grinding, homogenizing, suspending, dissolving, emulsifying, dispersing, and/or mixing selected extracts or ingredients derived therefrom, with selected excipient(s), carrier(s), adjuvant(s) or diluent(s). For instance, as exemplified hereinafter, samples comprising resveratrol glycosides and high levels of resveratrol can be obtained by using an ethanol extraction (see Example 1), a methanol extraction (Example 2), or an ethyl acetate extraction extraction (Example 3) of plant materials from Gnetum africanum.

A particular aspect concerns purification methods for obtaining a plant extract having anti-inflammatory activity. Suitable methods according to the invention include those known to those skilled in the art as “activity-guided purification” or “bioassay-guided purification”, i.e. for this particular case, using methods, techniques, and conditions supporting, and more preferably maximizing, extractions of anti-inflammatory active component(s).

In one embodiment the purification method of the invention comprises:
a) providing plant material from Gnetum africanum;
b) submitting the plant material to a solvent extraction; and
c) removing the solvent.

Preferably the plant material is from a stem of the plant Gnetum africanum. Suitable solvents include, but are not limited to, ethyl acetate, ethanol, and methanol.

In another embodiment the method comprises the steps of:
(i) grinding plant material (preferably previously dried) from Gnetum africanum;
(ii) extracting the ground plant material with one or more suitable solvents to obtain a solvent extract;
(iii) recovering and drying the solvent extract to obtain the plant extract.

The methods may further comprise the steps of ultrasonicating the plant material in presence of the one or more suitable solvents at the solvent extraction step. In one embodiment, obtaining or recovering the resveratrol-concentrated plant extract comprises a centrifugation step. It may be preferable to carry out two rounds of (1) removing a supernatant and (2) re-suspending the pellet after each centrifugation step, and the supernatants from the rounds of centrifugation are pooled and dried.

In preferred embodiments the solvent extraction is carried out with techniques and conditions supporting and/or maximizing extraction of known or potential anti-inflammatory active component(s), including but not limited to resveratrol, resveratrol glysoside-1 (Table 1), resveratrol glysoside-2 (Table 1), and combinations thereof.

### Methods of Uses

As indicated hereinbefore and exemplified hereinafter, the compounds and extracts according to the invention have beneficial therapeutic and pharmaceutical anti-inflammatory properties and therefore, may have useful pharmaceutical applications in the prevention and/or treatment of inflammation. Accordingly, the invention further contemplates methods of use and methods of treatment, comprising the use or administration of a therapeutically effective amount of a plant extract, a resveratrol, resveratrol glycoside and/or composition as defined herein.

As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc.), and the particular disorder(s) the subject is suffering from. In addition, the therapeutically effective amount may depend on the subject's condition, the severity of the disease state, or underlying disease or complications.

Appropriate and acceptable doses may be determined using many available techniques, including bioavailability studies and a range of clinical studies. When one or more of the compounds of the invention is to be administered to humans, a physician may for example, prescribe a relatively low dose at first, subsequently increasing the dose until an appropriate response is obtained.

According to preferred embodiments, the compounds and extracts defined herein are used for the prevention and/or treatment of an inflammatory-related disease a mammalian subject in need thereof. The term “mammalian subject” includes mammals in which inhibition of inflammation is desirable. The term “subject” includes domestic animals (e.g. cats, dogs, horses, pigs, cows, goats, sheep), rodents (e.g. mice or rats), rabbits, squirrels, bears, primates (e.g., chimpanzees, monkeys, gorillas, and humans), and transgenic species thereof. Preferably, the mammalian subject is a human, more preferably a human patient in need of treatment due to an inflammatory-related disease.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

As used herein, the terms “treatment” or “treating” of a subject include the administration of a compound of the invention to a subject with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

More particularly, methods of treatment according to the invention comprise the administration to a subject in need thereof of a therapeutically effective amount of a compound, composition or extract as defined herein. A related aspect concerns the use of a plant extract, resveratrol, a resveratrol glycoside and/or a composition as defined herein for the manufacture of a medicament for prevention or treatment of inflammatory diseases or related conditions.

Accordingly, a related aspect of the invention concerns a method for the in vivo treatment of inflammatory-related diseases in a human subject. In one embodiment, the method comprises administering to a human subject in need thereof a therapeutically effective amount of a compound and/or extract as defined herein.

The invention further concerns a method for the in vivo treatment of a skin-related diseases and cutaneous conditions in a human subject. Examples of skin-related diseases and cutaneous conditions encompassed by the invention include, but are not limited to, condition where there is an inflammation of the skin tissues. In one embodiment, the method comprises contacting the skin cells of a human subject in need thereof with a therapeutically effective amount of a compound and/or extract as defined herein.

The compounds and/or extracts of the invention may be administered with any conventional treatments, including currently available anti-inflammatory drugs and anti-inflammatory agents such as nonsteroidal anti-inflammatory drugs, acetaminophen, COX-2 inhibitors, acetic acid derivatives, propionic acid derivatives and fenamates. In order to evaluate, assess, and/or confirm the efficacy of the method, compounds and/or compositions of the invention, serial measurements can be determined. Quantitative methods and techniques for the assessment of inflammation are well known in the art.

### Pharmaceutical and Cosmetic Compositions

Related aspects of the invention concern cosmetic and pharmaceutical compositions comprising an effective amount compound and/or extract as defined herein. One particular aspect concerns the use of a therapeutically effective amount of compound and/or extract for the prevention or treatment of an inflammatory disease. Another particular aspect concerns the use of a compound and/or extract as defined herein in the manufacture of a medicament for the prevention or treatment of an inflammatory disease.

“Pharmaceutically acceptable vehicle” or “cosmetically acceptable vehicle” refers to a diluent, adjuvant, excipient, or carrier with which a compound is administered. The terms “pharmaceutically acceptable” and “cosmetically acceptable” refer to drugs, medicaments, inert ingredients, etc., which are suitable for use in contact with the skin tissues of humans and animals without undue toxicity, incompatibility, instability, irritation, allergic response, and the like, commensurate with a reasonable benefit/risk ratio. It preferably refers to a compound or composition that is approved or approvable by a regulatory agency of the Federal or state government or listed in the U.S. Pharmacopoeia or other generally recognized pharmacopoeia for use in animals and more particularly in humans. The pharmaceutically or cosmetically acceptable vehicle can be a solvent or dispersion medium containing, for example, water, ethanol, polyol (for example, glycerol, propylene glycol, and liquid polyethylene glycol and the like), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms in the composition can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

The compounds of the invention may be formulated prior to administration into pharmaceutical and/or cosmetic compositions using available techniques and procedures. Accordingly, another aspect of the invention concerns compositions and products comprising a plant extract, a resveratrol and/or a resveratrol glycoside as defined herein.

Typically the composition and products of the invention include active ingredients which are derived from Gnetum africanum. In a preferred embodiment, the composition comprises at least 0.002% w/w resveratrol derived from Gnetum africanum. In various embodiments, the composition may comprise from at least 0.0025% w/w, 0.003% w/w, 0.004% w/w, 0.005% w/w, 0.075% w/w, 0.01% w/w, 0.02% w/w, 0.05% w/w, 0.1% w/w, 0.5% w/w, 1% w/w or more resveratrol.

In preferred embodiments, the compositions according to the invention are formulated for oral administration. Additional routes of administration include intravenous (iv), intramuscular (im), depo-im, subcutaneous (sc), depo-sc, sublingually, intranasal, intrathecal, topical or rectal routes.

The methods of treatment of the present invention may also include co-administration of the at least one compound or extract according to the invention together with the administration of another therapeutically effective anti-inflammatory agent or drug. Examples of such anti-inflammatory agents include, but are not limited to, nonsteroidal anti-inflammatory drugs, acetaminophen, COX-2 inhibitors, acetic acid derivatives, propionic acid derivatives and fenamates.

In addition, the plant extracts, resveratrol, resveratrol glycosides and/or compositions of the invention may also contain additional ingredients, including but not limited to, metal chelators, metal scavengers, coating agents, preserving agents, solubilizing agents, stabilizing agents, wetting agents, emulsifiers, colorants (e.g. tracer dyes), odorants, salts, buffers, surfactants, solvents, coating agents and/or antioxidants.

For preparing the composition of the invention, methods well known in the art may be used. The compositions and extracts of the invention may be formulated under different suitable forms, which include, but are not limited to a dry powdered composition, tablets or capsules, a liquid solution, a suspension, a cream, an ointment, a lotion, a paste, etc. The compositions and extracts according to the invention may also be marketed as a concentrate to be diluted by an end user.

The plant extracts, resveratrol, resveratrol glycosides and/or compositions according to the invention may be packaged under different forms, such as a sealed container (e.g. a plastic or glass bottle, ampul, vial), a pouch (e.g. a bag or sachet), blister packs, etc.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further limiting.

### EXAMPLES

### Example 1

### Identification and Quantification of Resveratrol by HPLC-MS in Selected African Plants Following an Ethanol Extraction

### Experimental

The following samples of plant materials were received in dried unprocessed form: MO2, M03, M21, M23, E01, E08, E46, E47. The samples were dried leaves collected from Gnetum africanum growing either in Mayombe forest (M) or the equatorial forest (E) of Congo. The leaves were left to dry before extraction and analysis.

Plant Extraction: Plants were ground with a coffee blender, and stored as a powder at −80° C. until used. Each plant material was weighed (1g) into a 50 ml centrifuge tube and 20 mL 80% ethanol was pipetted into each tube for 1^{st} extraction. The sealed tubes were put into an ultrasonic processor for 30 min at room temperature (Yong-Jin Chao et al, (2006), Ultrasonication-assisted extraction of resveratrol from grapes, Journal of Food Engineering 77: 725-730). The supernatants were separated from the pellet after centrifugation at 3000×g for 10 min. Ten mL of 80% ethanol was added into the pellet and ultrasonication was repeated followed by centrifugation as above. Two supernatants were pooled into a weighted centrifuge tube and evaporated to dryness by a Speedvac™ at room temperature. The dried extracts were weighed and the percent yields were calculated.

HPLC-MSD Analysis: The HPLC-APCI-MS method for the analyses was modified from Saleem et a/. (Saleem A, Brendan R W, Harris C H, Muhammad A, Tayamo C, Sit S, Arnason J T. A validated method of phytochemical analysis of Flor-Essence™—A multiherb product. 2008. Phytochem Analysis Volume 20, Issue 5, pages 395-401). Briefly, an Agilent 1100™ Series HPLC-APCI-MSD system consisted of an autosampler, a quaternary pump, a column thermostat and a diode array detector. The mass spectrometric system, connected with HPLC system, was equipped with an atmospheric pressure chemical ionization (APCI) source, and a detector (range 50-1500 amu). All solvents were HPLC grade (Fisher Scientific, CA, USA) unless otherwise specified. For analytical scale HPLC-MS analysis the solvents were sonicated for 5 min and the column (YMC-ODS AM-C18™, 100×2.0 mm, particle size 3.0 micron, Waters Inc., CA, USA) was equilibrated for 15 min with the starting conditions prior to analysis. The elution solvent system consisted of A=acetonitrile and B=trifluoroacetic acid 0.05% (aqueous), pH 3.5. The optimal gradient elution conditions were 5-100% TFA over 20 min. The column was subsequently washed by increasing B up to 100% over 5 min and kept at isocratic condition for 5 min. The column was brought back to initial conditions in 5 min and equilibrated for 3 min. The total run time was 30 min. The column was operated at a flow of 0.25 ml/min and its temperature was maintained at 48 ° C. with maximum pressure limit of 200 bars.

The extracts were reconstituted to 1mg/ml in MeOH and filtered though 0.22 μm PTFE membranes respectively and 10 μL of each extract were injected into the HPLC system though an autosampler. The rest of the filtrate was stored at 4° C. as a reference sample. The UV spectra were stored at monitoring wavelengths of 280 nm. The mass spectrometer was operated in scan mode using positive polarity with mass detection range of 100-800 amu. The optimized conditions were, fragmentor voltage 80, gain 1.0 EMV, nitrogen gas temperature 150° C./350° C. in negative ionization mode, vaporizer 420° C., drying gas flow 5.0 l/min, nebulizer pressure 60 psig, capillary voltage 2100 V/4000V in negative ionization mode and corona at 25 μA/5 μA in negative ion mode. The quantification of the identified markers in the extracts was performed by calibrating with known concentration of standards on the basis of area under the peaks using diode array detection.

### Results

Plant Extraction: The extraction yields, using the ultrasonication-assisted extraction method, ranged from 8.7% in M03 to 20.2% in E01 (Table 3).

### LC-MS Analysis

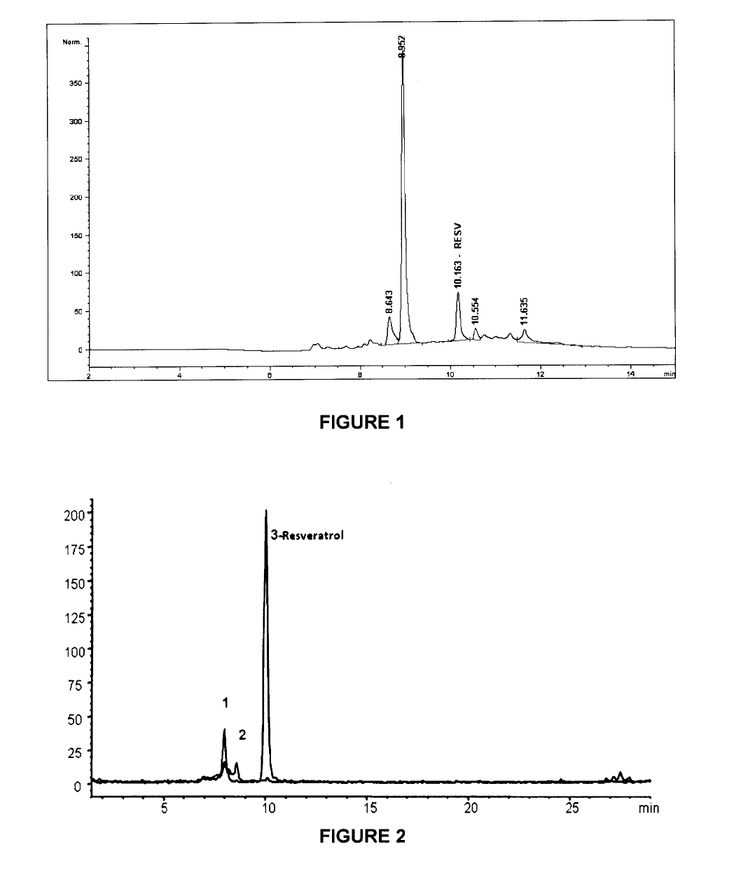

Identification of resveratrol: The confirmation of the presence of resveratrol was achieved by HPLC-DAD and LC-MSD analyses. The identification of resveratrol in plant extract chromatograms (FIG. 1) was confirmed by performing UV spectral match of the peak eluting at similar retention times with that of standard. Table 1 indicates the UV spectrometric characteristics of resveratrol obtained. The confirmation of the identification was further established by comparing the mass spectrometric fragmentation pattern of the standard resveratrol with the unknown peaks of the extracts eluting at the same time (FIG. 2). The two glycosides (1 and 2) were tentatively identified by MS (Table 2) eluting earlier than resveratrol. The chemical structure of these two resveratrol derivatives was not established.

TABLE 1
The identification of resveratrol and its derivatives by HPLC-
MSD

| # | Compound | Rt (min) | UV (280 nm) | MSD [M + H]⁺ |
|---|---|---|---|---|
| 1 | Resveratrol | 9.1 | 210 sh, 300, 310 sh | 229 [M + H]⁺, |
| | glycoside-1 | | | 391 [M + glyc]⁺ |
| 2 | Resveratrol | 9.5 | 210 sh, 300, 310 sh | 229 [M + H]⁺, |
| | glycoside-2 | | | 391 [M + glyc]⁺ |
| 3* | Trans-Resveratrol | 10.1 | 210 sh, 300, 310 sh | 229 [M + H]⁺ |

*confirmed by a reference standard

TABLE 2
Resveratrol and its derivatives in plant extracts determined by
HPLC-MSD

| | COMPOUND | | |
|---|---|---|---|
| | (see FIG. 2) | | |
| Plant extract | Glycoside 1 | Glycoside 2 | Resveratrol |
| E01 | | | ✓ |
| E08 | ✓ | ✓ | ✓ |
| E21 | | | ✓ |
| E46 | | | ✓ |
| E47 | ✓ | ✓ | ✓ |
| M02 | ✓ | | ✓ |
| M03 | | | ✓ |
| M23 | | | ✓ |

Quantification of resveratrol: A standard curve was generated by injecting 1 μL of five known concentrations of standard resveratrol, ranging from 500.0 μg/ml to 10.0 pg/ml dissolved in 100% methanol. As shown in Table 3 resveratrol is a predominant phytochemical and ubiquitously present in all extracts. The highest amount was found in E08 and M02 while it was detected in relatively lower amounts in E46, E47 and M21.

TABLE 3
Resveratrol content in selected plant extracts as determined by
HPLC-DAD.

| | | Resveratrol content in dried |
|---|---|---|
| Plant Extracts | Yield (%) * | plant material (μg/g) ** |
| E01 | 20.2 | 258.6 ± 5.07 |
| E08 | 16.5 | 2480 ± 185. |
| E46 | 15.2 | 67.0 ± 0.57 |
| E47 | 13.8 | 1442. ± 31.2 |
| M02 | 14.2 | 455.4 ± 1.83 |
| M03 | 8.7 | 133.8 ± 0.55 |
| M21 | 19.3 | 108.5 ± 10.2 |
| M23 | 15.5 | 187.4 ± 1.17 |

* The plant extracts were obtained after drying the supernatants to dryness by a rotary evaporator for 30-45 min. The water bath temperature was set at 40 degree Celsius. Yield was calculated as (weight of dried extract/weight of dried plant material) × 100
** The values are average values ± stdev, n = 3 and n = 5 for E01 and E08

### Example 2

### Identification of an Assay System to Determine Anti-Inflammatory Activity of Resveratrol and Gnetum africanum Extracts

Recently lyori et al., (Iyori M, Kataoka H, Shamsul H M, Kiura K, Yasuda M, Nakata T, Hasebe A, Shibata K. Resveratrol modulates phagocytosis of bacteria through an NF-kappaB-dependent gene program. Antimicrob Agents Chemother. 2008; 52:121-7) reported that resveratrol could inhibit LPS induced TNF-α production in RAW 264.7 cells. Accordingly, RAW 264.7 cells were treated with resveratrol (1 μM-100 μM) or G. africanum methanol extracts (25 μg/ml-200 μg/ml) for 1 hour, then the cells were induced with LPS (10 μg/ml) for 18 hr. Cell culture supernatants were used for the determination of TNF-α production by ELISA (R&D systems).

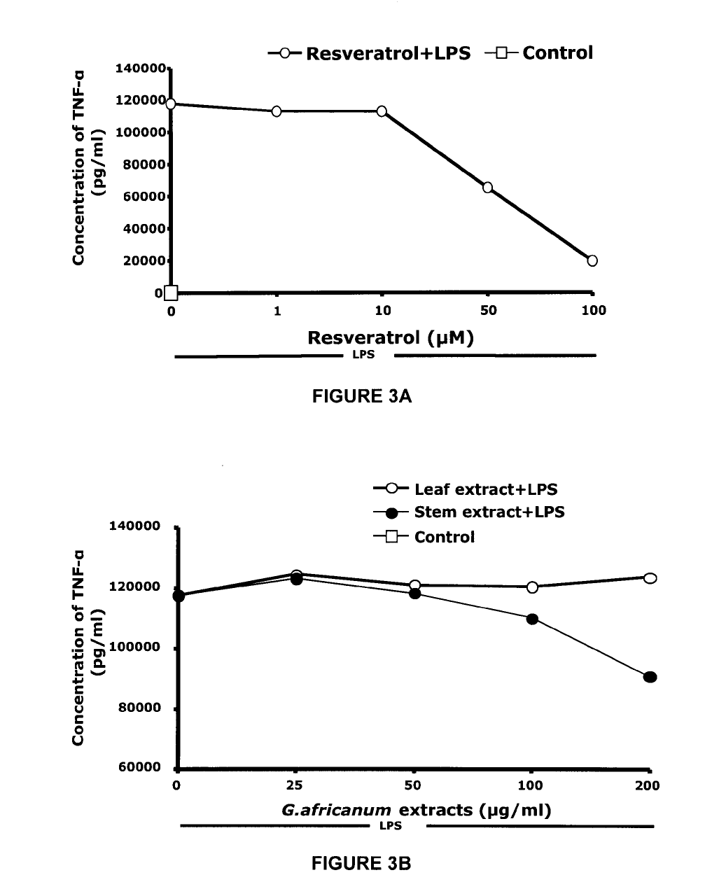

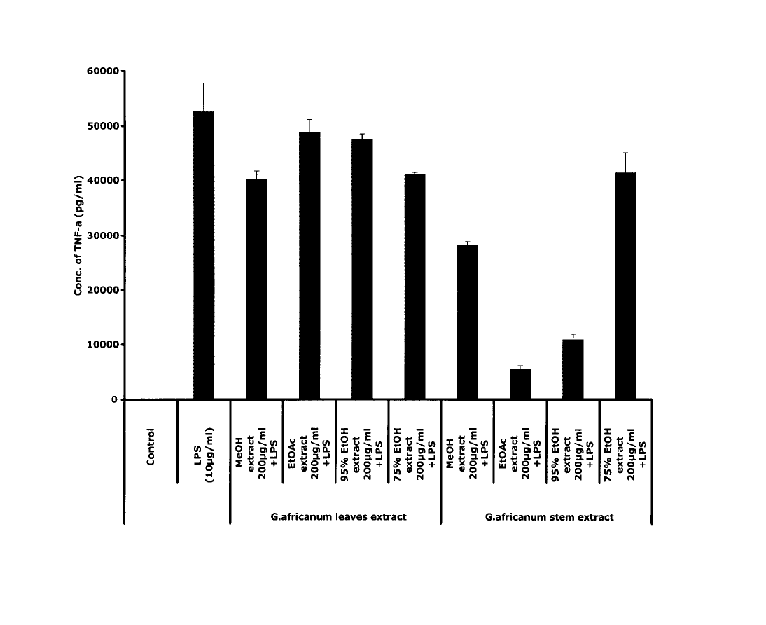

Resveratrol (at 50 μM and 100 μM concentrations) and Gnetum africanum stem extract (at 50 to 200 μg/ml concentrations) inhibited LPS-induced TNF-α production (FIG. 3A and 3B). Leaf extract treatment didn't show any inhibition even at 200 μg/ml. These results confirmed that the assay system was suitable for detecting the anti-inflammatory action of both resveratrol and the active components within the stem G. africanum extracts.

### Example 3

### Evaluation of Different Extraction Process for Anti-Inflammatory Activity

### Experimental and Results:

This experiment was designed to further investigate suitable solvent systems and extraction process to obtain anti-inflammatory activity in leaves and stem extracts derived from G. africanum. Various solvents were used to extract anti-inflammatory active components from freeze-dried plant material. The solvent details and their respective yields are given in the Table 4.

TABLE 4
Identification of suitable solvents for G. africanum extraction

| Samples | Solvent used | Yield |
|---|---|---|
| G. africanum leaves | Methanol | 19.4% |
| (Dried plant material) | 95% Ethanol | 13.3% |
| | 75% Ethanol | 18.5% |
| | Ethyl acetate | 4.6% |
| G. africanum stem | Methanol | 11.7% |
| (Dried plant material) | 95% Ethanol | 9.8% |
| | 75% Ethanol | 14.3% |
| | Ethyl acetate | 1.9% |

RAW 264.7 cells were treated with G. africanum leaf and stem extracts (200 μg/ml) for 1 hour and were then induced with LPS (10 μg/ml) for 18 hr. Cell culture supernatants were used for the determination of TNF-α production by ELISA (R&D systems). As shown in FIG. 2 stem extracts showed better anti-inflammatory activities than leaf extracts. The greatest anti-inflammatory activities were measured in the stem extract using Ethyl acetate (EtOAC) and 95% ethanol (EtOH).

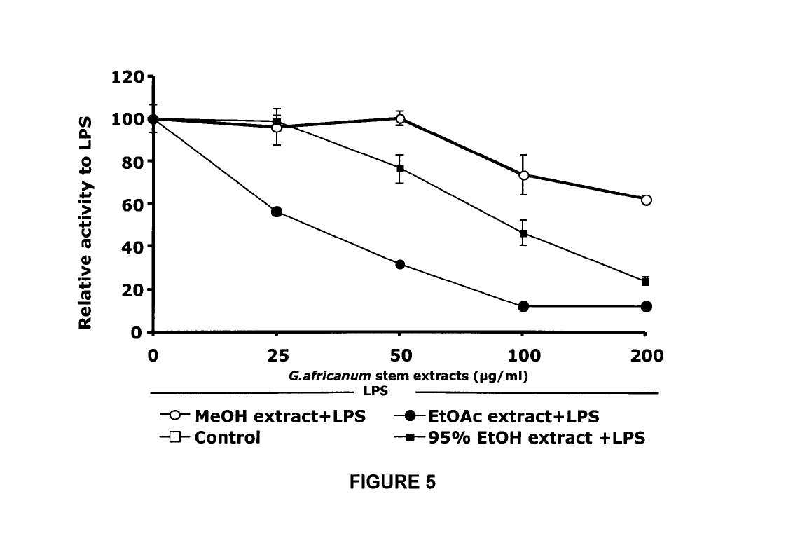

Methanol, ethyl acetate and 95% ethanol stem extracts were further analyzed in a dose dependent manner (25-200 μg/ml). As shown in FIG. 5, all extracts inhibited LPS-induced TNF-α production in RAW cells, with the greatest inhibition at the higher doses.

### Conclusions:

Among stem extracts, although the yield of the 95% ethanol extract was comparable to methanol extract (9.8% vs. 11.7%) the 95% ethanol extract was more active than methanol extract. This could either be due to the more effective extraction of active components by ethanol or that methanol may inactivate the active components during extraction. Table 5 hereinafter provides a comparison of the solvents tested for the stem extracts.

TABLE 5
Comparison of various solvents for active G. africanum stem
extracts

| Objective | Ranking |
|---|---|
| Anti-inflammatory | Ethyl acetate extract > 95% ethanol extract > |
| activity: | methanol extract |
| Yield: | Methanol extract ≧ 95% ethanol extract > |
| | ethyl acetate extract |

From FIG. 5 it is clear that that the IC₅₀ value of the ethyl acetate extract (25 μg/ml) is four times less than that of the 95% ethanol extract (100 μg/ml). However, the yield of the ethyl acetate extract is 5 times less than that of the ethanol extract (Table 4). This observation suggests that ethyl acetate is more selectively extracting active components from the G. africanum stem. In other words, ethyl acetate may be extracting the same amount of active components as 95% ethanol while the latter solvent is extracting more inactive components. For commercial purposes this means that 95% ethanol could be used for extraction instead of the more toxic ethyl acetate while still maintaining the quantitative and most likely complete extraction of active components.

On comparison, the anti-inflammatory activity of resveratrol (see FIG. 3) is IC₅₀=50 μM or 12 μg/ml while the IC₅₀ of the ethyl acetate stem extract is 30 μg/ml (FIG. 5). Since it is very unlikely that resveratrol makes up 40% of the ethyl acetate extract, this observation suggests that other components present in the stem extract are contributing to its anti-inflammatory potential in addition to resveratrol. Without being bound by any hypothesis, it is conceivable that resveratrol glycoside-1 and/or resveratrol glycoside-2 (see Table 1 and FIG. 2) play a role in the extracts' anti-inflammatory activity.

### Example 4

### Quantification of Biochemicals in Selected Parts of Gnetum africanum

Leaves and liana (stem) from Gnetum africanum growing either in Mayombe forest or the equatorial forest of Congo were analyzed for their biochemical content. A summary of the results are shown in Table 6.

TABLE 6
Biochemical analysis of Gnetum africanum

| | Forest of Mayombe | | Equatorial Forest | |
|---|---|---|---|---|
| | Leaves | Liana | Leaves | Liana |
| Protein content | 14.6% | 11.3% | 15.6% | 9.37% |
| Oil content | 3.2% | 3.1% | 6.4% | 1.6% |
| Omega 6 | 9.4% | 17.8% | | |
| Total trans fat | 29.8% | 39.4% | | |
| Cis fat | 12.4% | 21.1 | | |
| Omega 3 | 48.4 | 21.7 | | |
| Minerals (mg/100 g) | | | | |
| Bore (B) | 1.07 | 0.485 | N/A | N/A |
| Calcium (Ca) | 0.75 | 0.19 | 1.23 | 0.79 |
| Chrome (Cr) | 0.06 | 0.025 | N/A | N/A |
| Cobalt (Co) | 0.01 | 0.015 | N/A | N/A |
| Magnesium (Mg) | 43 | 32.49 | N/A | N/A |
| Manganese (Mn) | 12.15 | 4.58 | N/A | N/A |
| Iron (Fe) | 2.37 | 1.47 | N/A | N/A |
| Copper (Cu) | 0.63 | 1 | N/A | N/A |
| Nickel (Ni) | 0.055 | 0.05 | N/A | N/A |
| Phosphorus (P) | 44 | 49 | N/A | N/A |
| Potassium (K) | 1.45 | 1.44 | 0.62 | 1.02 |
| Sélénium (Se) | 50.49 | 49.15 | N/A | N/A |
| Sodium (Na) | 0.5 | 0.055 | 0.018 | 0.030 |
| Zinc (Zn) | 0.41 | 0.66 | N/A | N/A |
| Lithium (Li) | 1.8 | 3.7 | N/A | N/A |
| Aluminium (Al) | 10.61 | 17.2 | N/A | N/A |
| Silicium (Si) | 8 | 3.76 | N/A | N/A |
| Titane (Ti) | 0.21 | 0.145 | N/A | N/A |
| Strontium (Sr) | 0.88 | 1.31 | N/A | N/A |
| Antimoine (Sb) | 0.14 | 0.01 | N/A | N/A |
| Rubidium (Rb) | 1.4 | 2.56 | N/A | N/A |
| Total fibers | 66.5 | 81.9 | 48.6 | N/A |
| Soluble fibers | 0.8 | 0.1 | 10.7 | N/A |
| Insoluble fibers | 65.7 | 81.1 | 37.9 | N/A |
| Resveratrol (mg/100 g) | 20.8 | 18.2 | 15.8 | 250 |
| Total sugar | 5.8% | 2.0% | 6.5% | 6.7% |
| Vitamin E mg/100 g | 71.8 | 1.8 | 38.2 | <1 |

Headings are included herein for reference and to aid in locating certain sections These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a compound” includes one or more of such compounds, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A plant extract derived from Gnetum africanum, wherein said plant extract comprises an anti-inflammatory activity.

2. The plant extract of claim 1, wherein said plant extract derives from a stem of the plant Gnetum africanum.

3. (canceled)

4. The plant extract of claim 1, wherein said plant extract is dried and comprises at least about 30 μg resveratrol per gram of dried Gnetum africanum.

5. The plant extract of claim 1, wherein said plant extract comprises a resveratrol glycoside.

6. The plant extract of claim 5, wherein said resveratrol glycoside is selected from: (i) a compound identifiable by the presence of a peak having a retention time of 9.1 min on a HPLC-APCI-MSD chromatogram under detection conditions of Table 1; and (ii) a compound having a retention time of 9.5 min on a HPLC-APCI-MSD chromatogram under detection conditions of Table 1.

7. The plant extract of claim 1, wherein said anti-inflammatory activity is measurable by an in vitro assay assessing LPS-induced TNF-α production in RAW cells, and wherein said plant extract inhibits said LPS-induced TNF-α production.

8. A purification method for obtaining a plant extract having an anti-inflammatory activity, the method comprising:
(i) providing plant material from Gnetum africanum;
(ii) submitting said plant material to a solvent extraction; and
(iii) removing said solvent.

9. The method of claim 8, wherein said solvent extraction is carried out with techniques and conditions supporting extraction of an anti-inflammatory active component.

10. The method of claim 9, wherein said anti-inflammatory active component is selected from the group consisting of resveratrol, resveratrol glysoside-1, resveratrol glysoside-2, and combinations thereof.

11. The method of claim 9, wherein said solvent is selected from the group consisting of ethyl acetate, ethanol, and methanol.

12. A composition comprising: (i) a plant extract derived from Gnetum africanum and comprising an anti-inflammatory activity; (ii) a purified plant extract derived from Gnetum africanum and obtained by solvent extraction; and/or (iii) a resveratrol glycoside selected from the group consisting of resveratrol glysoside-1, resveratrol glysoside-2 and combinations thereof; in combination with a suitable carrier, diluent or excipient.

13. The composition of claim 12, wherein said composition further comprises an anti-inflammatory agent and/or an antioxidant.

14. A resveratrol glycoside which is selected from: (i) a compound identifiable under detection conditions of Table 1 by the presence of a peak having a retention time of 9.1 min on a HPLC-APCI-MSD chromatogram; and (ii) a compound identifiable under detection conditions of Table 1 by the presence of a peak having a retention time of 9.5 min on a HPLC-APCI-MSD chromatogram.

15. The resveratrol glycoside of claim 14, wherein said resveratrol glycoside is isolated or purified from Gnetum africanum.

16-20. (canceled)

21. A method for the prevention and/or treatment of an inflammatory disease, comprising administering to a subject in need thereof a composition comprising a suitable carrier, diluent or excipient and; (i) a plant extract derived from Gnetum africanum and comprising an anti-inflammatory activity; (ii) a purified plant extract derived from Gnetum africanum and obtained by solvent extraction; and/or (iii) a resveratrol glycoside selected from the group consisting of resveratrol glysoside-1, resveratrol glysoside-2 and combinations thereof.

22. The method of claim 21, wherein the inflammatory disease is selected from the group consisting of arthritis, erythematosus, glomerulonephritis, vasculitis, psoriatic arthritis, systemic lupus erythematosus (SLE), idiopathic thrombocytopenic purpura (ITP), psoriasis, Crohn's disease, inflammatory bowel disease, ankylosing spondylitis, Sjogren's syndrome, Still's disease, uveitis, scleroderma, myositis, Reiter's syndrome, and Wegener's syndrome.

23. (canceled)

24. The composition of claim 12, wherein said composition is a cosmetic composition and wherein said cosmetic composition is formulated as a dry powdered composition, a tablet, a capsule, a liquid solution, a suspension, a cream, an ointment, a lotion or a paste suitable for a cosmetic use.

25. The plant extract of claim 1, wherein said plant extract is dried and comprises at least about 100 μg resveratrol per gram of dried Gnetum africanum.

26. The plant extract of claim 1, wherein said plant extract is dried and comprises at least about 500 μg resveratrol per gram of dried Gnetum africanum.

27. The plant extract of claim 1, wherein said plant extract is dried and comprises at least about 1500 μg resveratrol per gram of dried Gnetum africanum.
